(19) United States
(12) Patent Application Publication
(10) Pub. No.: US 2026/0260752 A1
(43) Pub. Date: Sep. 3, 2026

(54) ENCODING MEDICAL DOMAIN KNOWLEDGE INTO PREDICTION MODEL TRAINING FOR FLOW CYTOMETRY-BASED DIAGNOSIS

(71) Applicant: UNIVERSITY OF UTAH RESEARCH FOUNDATION, Salt Lake City, UT (US)

(72) Inventors: Muir Morrison, Salt Lake City, UT (US); Mattia Medina-Grespan, Salt Lake City, UT (US); David P. Ng, Salt Lake City, UT (US); Vivek Srikumar, Salt Lake City, UT (US); Brendan Daniel O'Fallon, Salt Lake City, UT (US)

(21) Appl. No.: 19/544,882

(22) Filed: Feb. 19, 2026

Related U.S. Application Data
(60) Provisional application No. 63/765,264, filed on Feb. 28, 2025.

(51) Int. Cl. G16H 50/20; G01N 15/14

(52) U.S. Cl. CPC G16H 50/20; G01N 15/14

(57) ABSTRACT

Methods, systems, and apparatus for medical diagnosis using cytometry data. One method includes receiving training flow cytometry data and obtaining a superclass-subclass relationship between a plurality of diagnosis labels in a diagnosis label space, wherein a diagnosis label represents a medical condition based on the training flow cytometry data. The method also includes, via the electronic processor, encoding cytometry domain knowledge into a plurality of soft constraints including a relationship constraint representing a correlation between a pair of diagnosis labels of the plurality of diagnosis labels and a hierarchical constraint representing the superclass-subclass relationship and training a machine learning model using a loss function including the plurality of soft constraints. The method further includes receiving clinical flow cytometry data from a patient sample and generating one or more diagnosis labels of the plurality of diagnosis labels for the clinical flow cytometry data using the trained machine learning model.

### RELATED APPLICATIONS

This application claims priority to U.S. Provisional Application No. 63/765,264, filed Feb. 28, 2025, the entire content of which is incorporated herein by reference.

### FIELD

Examples described herein generally relate to medical diagnosis using machine learning based on flow cytometry data, and, in particular, to encoding medical domain knowledge into prediction model training for flow cytometry-based diagnosis.

### SUMMARY

Flow cytometry is a technique used in biology, immunology, and medical diagnostics to analyze physical and chemical characteristics of individual cells or particles as the cells or particles flow in a fluid stream through a beam of light. Flow cytometry technique involves rapid and simultaneous measurement of multiple parameters for the cells within a heterogeneous population, providing detailed information for diagnosing various blood disorders and immune system abnormalities.

Some machine learning approaches for flow cytometry data analysis use labeled datasets and process diagnoses as independent labels. The independent labels do not consider relationships between medical conditions. There are also technical difficulties in modifying neural network architectures to incorporate structured cytometry domain knowledge so that the relationships between the medical conditions are enforced. Additionally, there are technical difficulties in translating qualitative cytometry domain knowledge into quantitative constraints that can guide the training process. Also, there are technical difficulties in maintaining model flexibility while enforcing constraints where the relationships are encoded. Moreover, there are technical difficulties in designing a loss function that can optimize for prediction accuracy and maintain consistency with cytometry domain expert knowledge based on which the relationships between the medical conditions are derived.

Aspects of the present disclosure provide technical improvements and technical solutions to the above noted difficulties. Examples described herein employ a neuro-symbolic artificial intelligence (AI) approach that combines the power of neural networks with expert-driven symbolic logic. This hybrid approach distinguishes itself by incorporating expert knowledge during the model's training phase to guide predictions that align with established medical knowledge. Additionally, the systems and methods described herein enforces rules that ensure the structural consistency of the label space, leading to more accurate predictions, particularly for diagnoses with infrequent data representation. In other words, the systems and methods described herein integrate neural network-based learning with symbolic logic from expert knowledge to inform and guide predictions, wherein, unlike conventional supervised learning methods, the systems and methods flexibly incorporate external expert knowledge, compensating for the scarcity of labeled training examples, which is common in the diagnosis of rare medical conditions. By injecting the expert knowledge as soft constraints into the model's loss function during training, a more flexible and efficient learning process is achieved as compared to other approaches that apply hard constraints externally after the model is trained. The systems and methods provide consistency in the label space by implementing rules that maintain the structural consistency of the label space, which ensures that predicted diagnoses adhere to medically valid relationships between diagnosis labels. By integrating soft constraints during training, the systems and methods improve prediction accuracy, particularly for less frequent diagnoses, which are often underrepresented in labeled datasets.

In particular, methods and systems described herein leverage structural dependencies in the diagnosis label space, informed by expert knowledge, which compensates for the scarcity of labeled data that is often expensive and difficult to obtain (especially in the field of flow cytometry). Thus, the methods and systems reduce the need for large amounts of labeled training data, which lowers costs (in terms of time and computing resources) associated with data collection and annotation. Additionally, the systems and methods described herein establish a model with enhanced ability to effectively predict high-risk infrequent diagnoses, which improves the quality and reliability of diagnostic systems in medical application-including critical medical applications.

For example, examples described herein provide a neuro-symbolic architecture that combines neural networks with symbolic logic to process flow cytometry data while maintaining diagnostic relationships. Examples described herein encode domain knowledge as soft constraints and translate medical relationships into differentiable mathematical expressions. Examples also provide a modified loss function that incorporates both prediction accuracy and knowledge constraints, resulting in simultaneous optimization of both objectives. In addition, examples described herein provide a hierarchical constraint design that maintains superclass-subclass relationships between diagnoses while training the model using flow cytometry data. These technical solutions reduce dependence on labeled datasets and leverages expert knowledge regarding relationships between the medical conditions to improve predictions generated via the trained models.

### BRIEF DESCRIPTION OF THE DRAWINGS

FIG. 1 illustrates a cytometry knowledge-constraint medical diagnostic apparatus for processing flow cytometry data according to some examples.

FIG. 2 illustrates a process flow diagram showing steps for training and utilizing a machine learning model for medical diagnosis using flow cytometry data, all or a portion of which may be implemented by the apparatus of FIG. 1 according to some examples.

FIG. 3 illustrates a flowchart of an example method for medical diagnosis using flow cytometry data according to the medical diagnosis process of FIG. 2.

FIG. 4 illustrates a portion of a correlation matrix as one, non-limiting example.

### DETAILED DESCRIPTION

One or more examples are described and illustrated in the following description and accompanying drawings. These examples are not limited to the specific details provided herein and may be modified in various ways. Furthermore, other examples may exist that are not described herein. Also, the functionality described herein as being performed by one component may be performed by multiple components in a distributed manner. Likewise, functionality performed by multiple components may be consolidated and performed by a single component. Similarly, a component described as performing particular functionality may also perform additional functionality not described herein. For example, a device or structure that is “configured” in a certain way is configured in at least that way but may also be configured in ways that are not listed.

Furthermore, some examples described herein may include one or more electronic processors configured to perform the described functionality by executing instructions stored in non-transitory, computer-readable medium (e.g., to perform the computer-implemented methods described herein). Similarly, examples described herein may be implemented as non-transitory, computer-readable medium storing instructions executable by one or more electronic processors to perform the described functionality. As used in the present application, “non-transitory computer-readable medium” comprises all computer-readable media but does not consist of a transitory, propagating signal. Accordingly, non-transitory computer readable medium may include, for example, a hard disk, a CD-ROM, an optical storage device, a magnetic storage device, a ROM (Read Only Memory), a RAM (Random Access Memory), register memory, a processor cache, or any combination thereof.

Unless the context of their usage unambiguously indicates otherwise, the articles “a,” “an,” and “the” should not be interpreted as meaning “one” or “only one.” Rather these articles should be interpreted as meaning “at least one” or “one or more.” Likewise, when the terms “the” or “said” are used to refer to a noun previously introduced by the indefinite article “a” or “an,” “the” and “said” mean “at least one” or “one or more” unless the usage unambiguously indicates otherwise.

Also, it should be understood that the illustrated components, unless explicitly described to the contrary, may be combined or divided into separate software, firmware and/or hardware. For example, as noted above, instead of being located within and performed by a single electronic processor, logic and processing described herein may be distributed among multiple electronic processors. Similarly, one or more memory modules and communication channels or networks may be used even if examples described or illustrated herein have a single such device or element. Also, regardless of how they are combined or divided, hardware and software components may be located on the same computing device or may be distributed among multiple different devices. Accordingly, in the claims, if an apparatus, method, or system is claimed, for example, as including a controller, control unit, electronic processor, computing device, logic element, module, memory module, communication channel or network, or other element configured in a certain manner, for example, to perform multiple functions, the claim or claim element should be interpreted as meaning one or more of such elements where any one of the one or more elements is configured as claimed, for example, to make any one or more of the recited multiple functions, such that the one or more elements, as a set, perform the multiple functions collectively.

In addition, the phraseology and terminology used herein is for the purpose of description and should not be regarded as limiting. For example, the use of “including,” “containing,” “comprising,” “having,” and variations thereof herein is meant to encompass the items listed thereafter and equivalents thereof as well as additional items. The terms “connected” and “coupled” are used broadly and encompass both direct and indirect connecting and coupling. Further, “connected” and “coupled” are not restricted to physical or mechanical connections or couplings and can include electrical connections or couplings, whether direct or indirect. In addition, electronic communications and notifications may be performed using wired connections, wireless connections, or a combination thereof and may be transmitted directly or through one or more intermediary devices over various types of networks, communication channels, and connections. Moreover, relational terms, such as, for example, first and second, top and bottom, and the like may be used herein solely to distinguish one entity or action from another entity or action without necessarily requiring or implying any actual such relationship or order between such entities or actions.

The flow cytometry data may be obtained from a cytometer. A cytometer generally refers to an analytical instrument used to analyze physical and chemical characteristics of individual cells or particles in a fluid stream. The cytometer comprises components, such as, for example, (i) a fluidics system that transports and aligns cells in a single file through a laser beam, (ii) one or more lasers for illumination, (iii) a series of optical filters and mirrors to direct specific wavelengths of scattered and fluorescent light, (iv) multiple photodetectors, such as, for example, photomultiplier tubes to capture and quantify the light signals, and (v) a computer system for data acquisition and analysis. For example, as cells pass though the laser beam, the cytometer (e.g., the computer system process data captured via the photodetector(s)) measures forward scatter, side scatter, and fluorescence emissions from labeled cellular components. The cytometer as described herein may be used to perform flow cytometry, mass cytometry, or imaging cytometer and may include various types of detectors. For example, in addition or in place of the photodetectors noted above, the cytometer may include one or more Photomultiplier Tubes (PMTs), Avalanche Photodiodes (APDs), Complementary Metal Oxide Semiconductor (CMOs), Charged Coupled Devices (CCDs), or a combination thereof. In some examples, the functionality and methods described herein may be performed via the computer system of the cytometer, via one or more computer systems external to the cytometer, or a combination thereof.

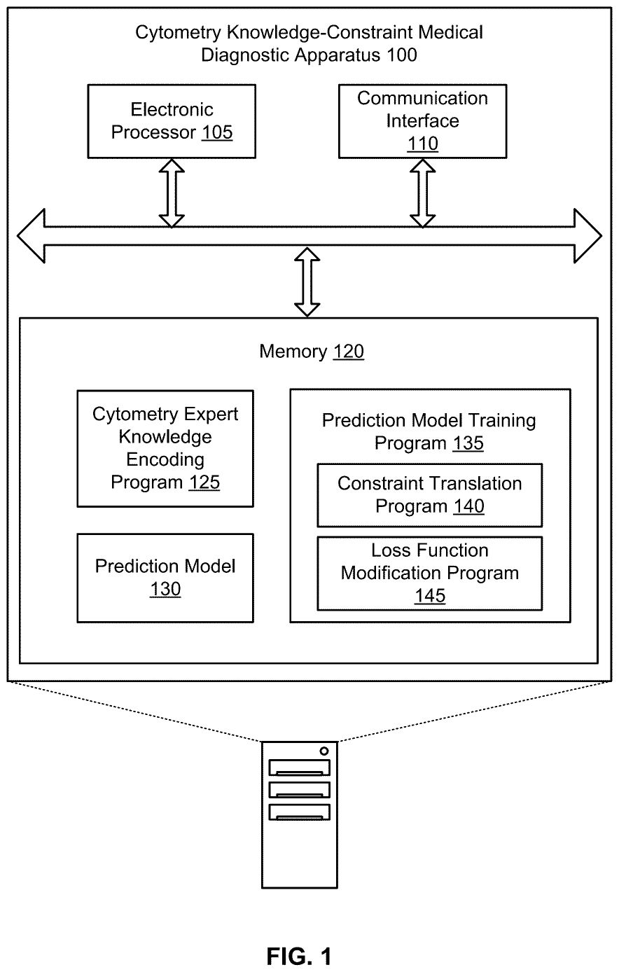

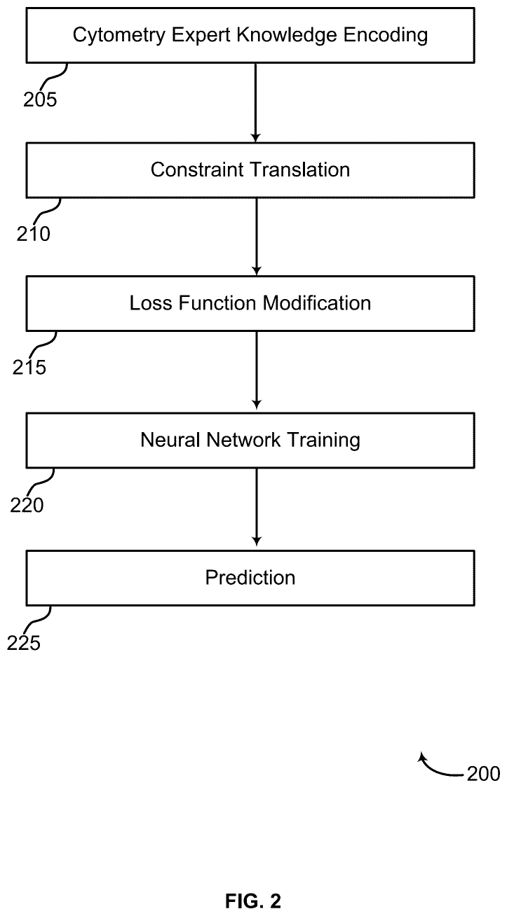

FIG. 1 illustrates a cytometry knowledge-constraint medical diagnostic apparatus 100 for processing flow cytometry data. FIG. 2 illustrates a process flow diagram showing steps for training and utilizing a machine learning model for medical diagnosis using flow cytometry data, all or a portion of which may be implemented by the apparatus 100.

The cytometry knowledge-constraint medical diagnostic apparatus 100 includes an electronic processor 105, a communication interface 110, and a memory 120. The electronic processor 105, the communication interface 110, and the memory 120 communicate over one or more control and/or data buses. It should be understood that FIG. 1 illustrates only one example of the apparatus 100, and the apparatus 100 may include additional or fewer components and may perform functions other than those explicitly described herein.

In some instances, the electronic processor 105 is implemented as a microprocessor with separate memory, such as the memory 120. In other instances, the electronic processor 105 may be implemented as a microcontroller (with memory 120 on the same chip). In other instances, the electronic processor 105 may be implemented using multiple processors. In addition, the electronic processor 105 may be implemented partially or entirely as, for example, a field-programmable gate array (FPGA), and application specific integrated circuit (ASIC), and the like and the memory 120 may not be needed or be modified accordingly. In the example illustrated, the memory 120 includes non-transitory, computer-readable memory that stores instructions that are received and executed by the electronic processor 105 to carry out functionality described herein. The memory 120 may include, for example, a program storage area and a data storage area. The program storage area and the data storage area may include combinations of different types of memory, such as read-only memory and random-access memory.

The communication interface 110 may include one or more ports (e.g., for receiving one or more wired cables or connections), transceivers, transmitters, receivers, or a combination thereof for communication with one or more devices or networks external to the apparatus 100, such as, for example, one or more cytometers, other types of analytical instructions, user devices, display or output devices, or combinations thereof.

As illustrated in FIG. 1, the memory 120 includes a cytometry expert knowledge encoding program 125, a prediction model 130, and a prediction model training program 135 comprising a constraint translation program 140 and a loss function modification program 145.

The cytometry expert knowledge encoding program 125, when executed by the electronic processor 105, encodes expert knowledge about relationships between medical diagnoses into formal representations, including relationships between diagnosis labels and superclass-subclass relationships between diagnoses. A relationship between diagnosis labels may refer to clinically observed correlations between different medical conditions, such as when two conditions occur together or when a presence of one condition indicates increased likelihood of the presence of another condition. In some instances, the cytometry expert knowledge encoding program 125 represents the correlations between medical conditions using a correlation matrix, where each element in the matrix indicates the strength of relationship between the diagnoses of a pair of medical conditions. The superclass-subclass relationships may refer to hierarchical relationships between broader diagnostic categories and the respective, more specific subtypes, such as the relationship between leukemia as a superclass and acute lymphoblastic leukemia as a subclass. The hierarchical relationships may be represented using a tree structure where parent nodes represent broader diagnostic categories and child nodes represent the respective, more specific diagnoses.

In some instances, the cytometry expert knowledge encoding program 125 assigns numerical weights to a plurality of relationships to indicate the relative importance in the diagnostic process. In some instances, the cytometry expert knowledge encoding program 125 validates the encoded relationships to ensure consistency with established medical knowledge and formats the encoded knowledge for subsequent processing by the constraint translation program 140. The cytometry expert knowledge encoding performed by the cytometry expert knowledge encoding program 125 is illustrated as the cytometry expert knowledge encoding step 205 in FIG. 2.

The constraint translation program 140, when executed by the electronic processor 105, translates the encoded expert knowledge into mathematical expressions that can be incorporated into the model's loss function as soft constraints. A hard constraint is a constraint that is strictly satisfied. A soft constraint, in contrast to the hard constraint, allows for flexibility in satisfying the encoded relationships while still guiding the model's learning process. In some instances, the constraint translation program 140 converts the correlation matrix elements into differentiable loss terms that penalize predictions violating the encoded relationships. In some instances, the constraint translation program 140 translates hierarchical relationships into inequality constraints so that the predicted probability of a subclass diagnosis does not exceed that of a respective superclass. The constraint translation performed by the constraint translation program 140 is illustrated as the constraint translation step 210 in FIG. 2.

The loss function modification program 145, when executed by the electronic processor 105, incorporates the translated constraints into the model's loss function. The base loss function may include cross-entropy loss measuring the accuracy of predicted diagnoses compared to true labels. The program adds the translated soft constraints to this base loss, with each constraint type weighted according to its importance. The modified loss function L may take the form L=CE (y,ŷ)+λ₁C₁(ŷ)+λ₂C₂(ŷ), where CE represents cross-entropy loss, C₁ and C₂ represent correlation and hierarchical constraint terms respectively, y represents the ground truth diagnosis labels, ŷ represents the predicted diagnosis labels output by the model, and λ₁, λ₂ are weights. The λ₁, λ₂ may be learnable or pre-determined. The modified loss function L guides the model to make predictions that are both accurate and consistent with expert knowledge. The loss function modification performed by the loss function modification program 145 is illustrated as the loss function modification step 215 in FIG. 2.

The prediction model training program 135, when executed by the electronic processor 105, performs the training process for the prediction model 130. The prediction model training program 135 may coordinate between the constraint translation program 140 and the loss function modification program 145 to implement neural network training. The training process involves iteratively updating the model's parameters using the training flow cytometry data (e.g., received from one or more cytometers, such data also referred to herein as a training dataset). During each iteration, the prediction model training program 135 computes both the prediction loss and constraint-based losses (such as C₁ and C₂ in this example), combines the prediction loss and constraint-based losses based on the respective weights, and updates the model parameters to minimize the output of the modified loss function L. The training process performed by the prediction model training program 135 is illustrated as the neural network training step 220 in FIG. 2.

The prediction model 130 may comprise a neural network configured to receive flow cytometry data as input and predict (output) diagnosis labels based on the input data. Flow cytometry data may include multiple measurements of cellular characteristics, including cell size, granularity, and the presence of specific cellular markers. The prediction model 130 processes these multi-dimensional measurements through multiple neural network layers, where each layer applies learned weights and biases to transform input features into increasingly abstract representations. The output layer of the neural network includes probability distributions over a plurality of diagnosis labels. The plurality of diagnosis labels is in a diagnosis label space. The prediction model 130 performs diagnosis prediction for new, clinical flow cytometry data as illustrated in the prediction step 225 in FIG. 2.

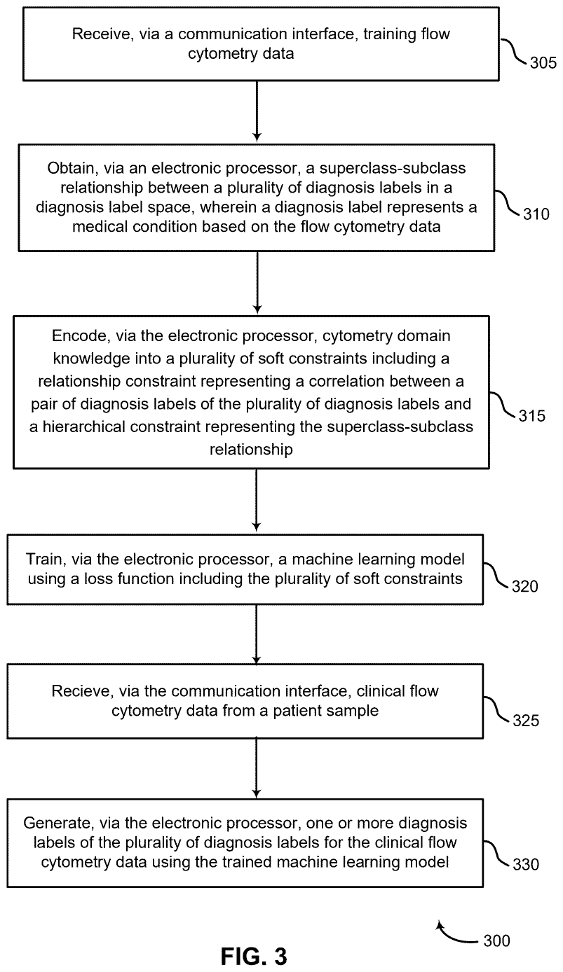

FIG. 3 illustrates a flowchart of an example method 300 for medical diagnosis using flow cytometry data according to the medical diagnosis process 200 shown in FIG. 2. The example method 300 may be implemented, in its entirety or at least a portion thereof, on the components of the cytometry knowledge-constraint medical diagnostic apparatus 100 shown in FIG. 1.

At operation 305, the cytometry knowledge-constraint medical diagnostic apparatus 100 receives, via the communication interface 110, training flow cytometry data. In some instances, the training flow cytometry data includes multi-dimensional measurements of cellular characteristics from patient samples with ground-truth diagnoses. For example, the flow cytometry data includes measurements of cell size, granularity, and various cell surface markers used for identifying cell types and conditions.

At operation 310, the cytometry knowledge-constraint medical diagnostic apparatus 100, obtains, via an electronic processor, a superclass-subclass relationship between a plurality of diagnosis labels in a diagnosis label space, wherein a diagnosis label represents a medical condition based on the training flow cytometry data. In some examples, the superclass-subclass relationship is between a first label representing a first medical condition as a superclass and a second label representing a second medical condition as a subclass, wherein a presence of the second medical condition indicates a presence of the first medical condition.

At operation 315, the cytometry knowledge-constraint medical diagnostic apparatus 100 encodes, via the electronic processor 105, cytometry domain knowledge into a plurality of soft constraints including a relationship constraint representing a correlation between a pair of diagnosis labels of the plurality of diagnosis labels and a hierarchical constraint representing the superclass-subclass relationship. In some examples, encoding the cytometry domain knowledge comprises translating a rule representing a relationship between the plurality of diagnosis labels into a differentiable mathematical expression, wherein the rule is obtained based on the cytometry domain knowledge, and the plurality of soft constraints include the differentiable mathematical expression. In some examples, the correlation between the pair of diagnosis labels is represented by a correlation matrix.

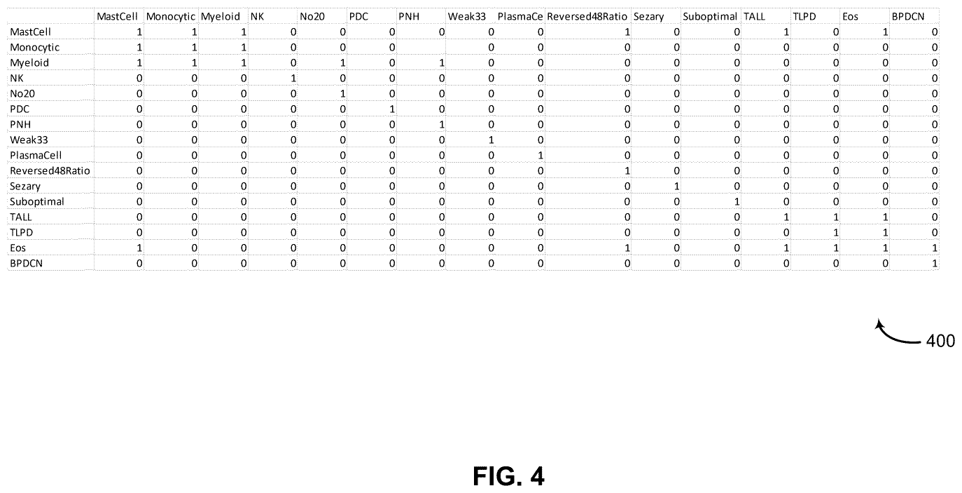

For example, the correlation between the pair of diagnosis labels can be represented by a correlation matrix where, if there are N diagnosis labels, the constraint matrix includes a N×N matrix where any off-diagonal element that has a predetermined value (e.g., a non-zero value) indicates that pair of diagnoses is more or less likely to co-occur than random chance (e.g., according to expert knowledge). Accordingly, for each element with the predetermined value (e.g., a non-zero value), the apparatus 100 (software program) would generate forward and reverse implications to add to the loss, pushing the model to either increase or decrease the co-occurrence of predictions of those labels. It should be understood that while a two-way implication is described, a one-way implication is also possible. FIG. 4 illustrates a portion of a correlation matrix 400 as one, non-limiting example.

The correlation matrix may be encoded as a dense matrix (e.g., as illustrated in FIG. 4) or a sparse encoding of pairwise soft associations. One or more hierarchical rules, type rules, or a combination thereof can be used with or separate from the correlation matrix or similar encoding. For example, below are example hierarchy constraints. The hierarchy constraints may identify diagnoses that are a superclass of the listed subcases. For example, in the below example, ‘N_BNHL’ is a superclass and (‘10+BNHL’, ‘5+BNHL’, ‘5-10-BNHL’, ‘B-NHL’, ‘BALL’, ‘CLL’, ‘DLBCL’, ‘HCL’, ‘Hematogones’, ‘No20’, ‘PlasmaCell’) is the list of subcases included in this superclass.

```
hierarchy_constraints = {
  ‘N_BNHL’: (‘10+BNHL’,‘5+BNHL’, ‘5-10-BNHL’, ‘B-NHL’, ‘BALL’, ‘CLL’, ‘DLBCL’,‘HCL’,
‘Hematogones’, ‘No20’, ‘PlasmaCell’),
  ‘N_TLPD’: (‘DE’, ‘GD_Tcells’, ‘High48Ratio’, ‘LGL’,‘NK’, ‘Reversed48Ratio’, ‘Sezary’, ‘TALL’,
‘TLPD’),
  ‘N_Myeloid’: (‘AML’, ‘Eos’, ‘Left Shift’, ‘MastCell’, ‘Monocytic’, ‘Myeloid’,‘PDC’, ‘PNH’,
‘Weak33’,‘BPDCN’),
  ‘N_Quality’: (‘Hemodilute’, ‘Paucicellular’, ‘Suboptimal’, ‘Clotted’),
  ‘N_AL’: (‘AML’, ‘BALL’, ‘TALL’),
}
```

Below are also example type rules. The type rules define diagnoses that never or rarely occur for a particular type. For example, the below example type rules indicate that “Hemodilute” and “Plasma Cell” are impossible or rare in peripheral blood, and “LeftShift” is impossible or rare in bone marrow.

```
type_rules = [
  ((‘TYPE’, “PERIPHERAL BLOOD”), (‘Hemodilute’, False))
  ((‘TYPE’, “PERIPHERAL BLOOD”), (‘PlasmaCell’, False)),
  ((‘TYPE’, “BONE MARROW”), (‘Left Shift’, False)),
]
```

At operation 320, the cytometry knowledge-constraint medical diagnostic apparatus 100 trains, via the electronic processor, a machine learning model using a loss function including the plurality of soft constraints. In some examples, the relationship constraint penalizes a prediction different from a correlation from a clinical observation; and the hierarchical constraint penalizes a prediction indicating a probability of a subclass diagnosis exceeds a probability of a respective superclass diagnosis. In some examples, the loss function includes a prediction loss term measuring difference between a predicted diagnosis label and ground truth, and wherein the machine learning model is trained based on the prediction loss term and the plurality of soft constraints.

At operation 325, the cytometry knowledge-constraint medical diagnostic apparatus 100 receives, via the communication interface 110, clinical flow cytometry data from a patient sample. For example, the clinical flow cytometry data includes real-time patient measurements obtained during clinical diagnosis. The clinical flow cytometry data includes the same types of cellular measurements as training flow cytometry data such as cell size, granularity, and cell surface markers. In this example, the clinical flow cytometry data are from patients without diagnosis and are unlabeled.

At operation 330, the cytometry knowledge-constraint medical diagnostic apparatus 100 generates, via the electronic processor, one or more diagnosis labels of the plurality of diagnosis labels for the clinical flow cytometry data using the trained machine learning model. In some examples, the predicted one or more diagnosis labels are in a diagnosis label space, and the plurality of diagnosis labels based on which the superclass-subclass relationships are derived during the operation 310 are in the same diagnosis label space.

Accordingly, examples described herein provide a neuro-symbolic architecture that combines neural networks with symbolic logic to process flow cytometry data while maintaining diagnostic relationships. Examples described herein encode domain knowledge as soft constraints and translate medical relationships into differentiable mathematical expressions. Examples also provide a modified loss function that incorporates both prediction accuracy and knowledge constraints, resulting in simultaneous optimization of both objectives. In addition, examples described herein provide a hierarchical constraint design that maintains superclass-subclass relationships between diagnoses while training the model using flow cytometry data. These technical solutions reduce dependence on labeled datasets and leverages expert knowledge regarding relationships between the medical conditions to improve predictions generated via the trained models. Thus, examples described herein provide a technological improvement in flow cytometry data analysis technology by, for example, providing an improvement over other computer-implemented analysis including other AI approaches. As previously noted, the systems and methods described herein integrate neural network-based learning with symbolic logic from expert knowledge to inform and guide predictions, wherein, unlike conventional supervised learning methods, the systems and methods flexibly incorporate external expert knowledge, compensating for the scarcity of labeled training examples, which is common in the diagnosis of rare medical conditions. By injecting the expert knowledge as soft constraints into the model's loss function during training, a more flexible and efficient learning process is achieved as compared to other approaches that apply hard constraints externally after the model is trained. The systems and methods provide consistency in the label space by implementing rules that maintain the structural consistency of the label space, which ensures that predicted diagnoses adhere to medically valid relationships between diagnosis labels. By integrating soft constraints during training, the systems and methods improve prediction accuracy, particularly for less frequent diagnoses, which are often underrepresented in labeled datasets.

In particular, methods and systems described herein leverage structural dependencies in the diagnosis label space, informed by expert knowledge, which compensates for the scarcity of labeled data that is often expensive and difficult to obtain (especially in the field of flow cytometry). Thus, the methods and systems reduce the need for large amounts of labeled training data, which lowers costs (in terms of time and computing resources) associated with data collection and annotation. Additionally, the systems and methods described herein establish a model with enhanced ability to effectively predict high-risk infrequent diagnoses, which improves the quality and reliability of diagnostic systems in medical application-including critical medical applications.

The following paragraphs provide various examples disclosed herein.

Example 1. A computer-implemented method for medical diagnosis using cytometry data, comprising: receiving, via a communication interface, training flow cytometry data; obtaining, via an electronic processor, a superclass-subclass relationship between a plurality of diagnosis labels in a diagnosis label space, wherein a diagnosis label represents a medical condition based on the training flow cytometry data; encoding, via the electronic processor, cytometry domain knowledge into a plurality of soft constraints including a relationship constraint representing a correlation between a pair of diagnosis labels of the plurality of diagnosis labels and a hierarchical constraint representing the superclass-subclass relationship; training, via the electronic processor, a machine learning model using a loss function including the plurality of soft constraints; receiving, via the communication interface, clinical flow cytometry data obtained from one or more cytometers from a patient sample; and generating, via the electronic processor, one or more diagnosis labels of the plurality of diagnosis labels for the clinical flow cytometry data using the trained machine learning model.

Example 2. The computer-implemented method of example 1, wherein encoding the cytometry domain knowledge comprises: translating a rule representing a relationship between the plurality of diagnosis labels into a differentiable mathematical expression, wherein the rule is obtained based on the cytometry domain knowledge, and the plurality of soft constraints include the differentiable mathematical expression.

Example 3. The computer-implemented method of example 1, wherein the superclass-subclass relationship is between a first label representing a first medical condition as a superclass and a second label representing a second medical condition as a subclass, wherein a presence of the second medical condition indicates a presence of the first medical condition.

Example 4. The computer-implemented method of example 1, wherein the one or more diagnosis labels are from the diagnosis label space.

Example 5. The computer-implemented method of example 1, wherein: the relationship constraint penalizes a prediction different from a correlation from a clinical observation; and the hierarchical constraint penalizes a prediction indicating a probability of a subclass diagnosis exceeds a probability of a respective superclass diagnosis.

Example 6. The computer-implemented method of example 3, wherein the loss function includes a prediction loss term measuring difference between a predicted diagnosis label and ground truth, and wherein the machine learning model is trained based on the prediction loss term and the plurality of soft constraints.

Example 7. The computer-implemented method of example 1, wherein the correlation between the pair of diagnosis labels is represented by a correlation matrix.

Example 8. A medical diagnostic apparatus, comprising: a communication interface configured to receive training flow cytometry data from a training dataset and clinical flow cytometry data obtained from one or more cytometers from a patient sample; and an electronic processor configured to: obtain a superclass-subclass relationship between a plurality of diagnosis labels in a diagnosis label space, wherein a diagnosis label represents a medical condition based on the training flow cytometry data, encode cytometry domain knowledge into a plurality of soft constraints including a relationship constraint representing a correlation between a pair of diagnosis labels of the plurality of diagnosis labels and a hierarchical constraint representing the superclass-subclass relationship, train a machine learning model a loss function including the plurality of soft constraints, and generate, using the trained machine learning model, one or more diagnosis labels of the plurality of diagnosis labels for the clinical flow cytometry data.

Example 9. The medical diagnostic apparatus of example 8, wherein to encode the cytometry domain knowledge, the electronic processor is configured to: translate a rule representing a relationship between the plurality of diagnosis labels into a differentiable mathematical expression, wherein the rule is obtained based on the cytometry domain knowledge, and the plurality of soft constraints include the differentiable mathematical expression.

Example 10. The medical diagnostic apparatus of example 8, wherein the superclass-subclass relationship is between a first label representing a first medical condition as a superclass and a second label representing a second medical condition as a subclass, wherein a presence of the second medical condition indicates a presence of the first medical condition.

Example 11. The medical diagnostic apparatus of example 8, wherein the one or more diagnosis labels are from the diagnosis label space.

Example 12. The medical diagnostic apparatus of example 8, wherein: the relationship constraint penalizes a prediction different from a correlation from a clinical observation; and the hierarchical constraint penalizes a prediction indicating a probability of a subclass diagnosis exceeds a probability of a respective superclass diagnosis.

Example 13. The medical diagnostic apparatus of example 8, wherein: the loss function includes a prediction loss term measuring difference between a predicted diagnosis label and ground truth, and wherein machine learning model is trained based on the prediction loss term and the plurality of soft constraints.

Example 14. The medical diagnostic apparatus of example 8, wherein the correlation between the pair of diagnosis labels is represented by a correlation matrix.

Example 15. A non-transitory computer-readable medium storing instructions that, when executed by an electronic processor, cause the electronic processor to: obtain a superclass-subclass relationship between a plurality of diagnosis labels in a diagnosis label space, wherein a diagnosis label represents a medical condition based on training flow cytometry data; encode cytometry domain knowledge into a plurality of soft constraints including a relationship constraint representing a correlation between a pair of diagnosis labels of the plurality of diagnosis labels and a hierarchical constraint representing the superclass-subclass relationship; train a machine learning model a loss function including the plurality of soft constraints; and generate, using the trained machine learning model, one or more diagnosis labels of the plurality of diagnosis labels for clinical flow cytometry data obtained from one or more cytometers.

Example 16. The non-transitory computer-readable medium of example 15, wherein the superclass-subclass relationship is between a first label representing a first medical condition as a superclass and a second label representing a second medical condition as a subclass, wherein a presence of the second medical condition indicates a presence of the first medical condition.

Example 17. The non-transitory computer-readable medium of example 15, wherein the one or more diagnosis labels are from the diagnosis label space.

Example 18. The non-transitory computer-readable medium of example 15, wherein: the relationship constraint penalizes a prediction different from a correlation from a clinical observation; and the hierarchical constraint penalizes a prediction indicating a probability of a subclass diagnosis exceeds a probability of a respective superclass diagnosis.

Example 19. The non-transitory computer-readable medium of example 15, wherein: the loss function includes a prediction loss term measuring difference between a predicted diagnosis label and ground truth, and wherein machine learning model is trained based on the prediction loss term and the plurality of soft constraints.

Example 20. The non-transitory computer-readable medium of example 15, wherein the correlation between the pair of diagnosis labels is represented by a correlation matrix.

Various features and advantages are set forth in the following claims.

What is claimed is:

1. A computer-implemented method for medical diagnosis using cytometry data, comprising:
receiving, via a communication interface, training flow cytometry data;
obtaining, via an electronic processor, a superclass-subclass relationship between a plurality of diagnosis labels in a diagnosis label space, wherein a diagnosis label represents a medical condition based on the training flow cytometry data;
encoding, via the electronic processor, cytometry domain knowledge into a plurality of soft constraints including a relationship constraint representing a correlation between a pair of diagnosis labels of the plurality of diagnosis labels and a hierarchical constraint representing the superclass-subclass relationship;
training, via the electronic processor, a machine learning model using a loss function including the plurality of soft constraints;
receiving, via the communication interface, clinical flow cytometry data obtained from one or more cytometers from a patient sample; and
generating, via the electronic processor, one or more diagnosis labels of the plurality of diagnosis labels for the clinical flow cytometry data using the trained machine learning model.

2. The computer-implemented method of claim 1, wherein encoding the cytometry domain knowledge comprises:
translating a rule representing a relationship between the plurality of diagnosis labels into a differentiable mathematical expression, wherein the rule is obtained based on the cytometry domain knowledge, and the plurality of soft constraints include the differentiable mathematical expression.

3. The computer-implemented method of claim 1, wherein the superclass-subclass relationship is between a first label representing a first medical condition as a superclass and a second label representing a second medical condition as a subclass, wherein a presence of the second medical condition indicates a presence of the first medical condition.

4. The computer-implemented method of claim 1, wherein the one or more diagnosis labels are from the diagnosis label space.

5. The computer-implemented method of claim 1, wherein:
the relationship constraint penalizes a prediction different from a correlation from a clinical observation; and
the hierarchical constraint penalizes a prediction indicating a probability of a subclass diagnosis exceeds a probability of a respective superclass diagnosis.

6. The computer-implemented method of claim 3, wherein the loss function includes a prediction loss term measuring difference between a predicted diagnosis label and ground truth, and wherein the machine learning model is trained based on the prediction loss term and the plurality of soft constraints.

7. The computer-implemented method of claim 1, wherein the correlation between the pair of diagnosis labels is represented by a correlation matrix.

8. A medical diagnostic apparatus, comprising:
a communication interface configured to receive training flow cytometry data from a training dataset and clinical flow cytometry data obtained from one or more cytometers from a patient sample; and
an electronic processor configured to:
obtain a superclass-subclass relationship between a plurality of diagnosis labels in a diagnosis label space, wherein a diagnosis label represents a medical condition based on the training flow cytometry data,
encode cytometry domain knowledge into a plurality of soft constraints including a relationship constraint representing a correlation between a pair of diagnosis labels of the plurality of diagnosis labels and a hierarchical constraint representing the superclass-subclass relationship,
train a machine learning model a loss function including the plurality of soft constraints, and
generate, using the trained machine learning model, one or more diagnosis labels of the plurality of diagnosis labels for the clinical flow cytometry data.

9. The medical diagnostic apparatus of claim 8, wherein to encode the cytometry domain knowledge, the electronic processor is configured to:
translate a rule representing a relationship between the plurality of diagnosis labels into a differentiable mathematical expression, wherein the rule is obtained based on the cytometry domain knowledge, and the plurality of soft constraints include the differentiable mathematical expression.

10. The medical diagnostic apparatus of claim 8, wherein the superclass-subclass relationship is between a first label representing a first medical condition as a superclass and a second label representing a second medical condition as a subclass, wherein a presence of the second medical condition indicates a presence of the first medical condition.

11. The medical diagnostic apparatus of claim 8, wherein the one or more diagnosis labels are from the diagnosis label space.

12. The medical diagnostic apparatus of claim 8, wherein:
the relationship constraint penalizes a prediction different from a correlation from a clinical observation; and
the hierarchical constraint penalizes a prediction indicating a probability of a subclass diagnosis exceeds a probability of a respective superclass diagnosis.

13. The medical diagnostic apparatus of claim 8, wherein:
the loss function includes a prediction loss term measuring difference between a predicted diagnosis label and ground truth, and wherein machine learning model is trained based on the prediction loss term and the plurality of soft constraints.

14. The medical diagnostic apparatus of claim 8, wherein the correlation between the pair of diagnosis labels is represented by a correlation matrix.

15. A non-transitory computer-readable medium storing instructions that, when executed by an electronic processor, cause the electronic processor to:
obtain a superclass-subclass relationship between a plurality of diagnosis labels in a diagnosis label space, wherein a diagnosis label represents a medical condition based on training flow cytometry data;
encode cytometry domain knowledge into a plurality of soft constraints including a relationship constraint representing a correlation between a pair of diagnosis labels of the plurality of diagnosis labels and a hierarchical constraint representing the superclass-subclass relationship;
train a machine learning model a loss function including the plurality of soft constraints; and
generate, using the trained machine learning model, one or more diagnosis labels of the plurality of diagnosis labels for clinical flow cytometry data obtained from one or more cytometers.

16. The non-transitory computer-readable medium of claim 15, wherein the superclass-subclass relationship is between a first label representing a first medical condition as a superclass and a second label representing a second medical condition as a subclass, wherein a presence of the second medical condition indicates a presence of the first medical condition.

17. The non-transitory computer-readable medium of claim 15, wherein the one or more diagnosis labels are from the diagnosis label space.

18. The non-transitory computer-readable medium of claim 15, wherein:
the relationship constraint penalizes a prediction different from a correlation from a clinical observation; and
the hierarchical constraint penalizes a prediction indicating a probability of a subclass diagnosis exceeds a probability of a respective superclass diagnosis.

19. The non-transitory computer-readable medium of claim 15, wherein:
the loss function includes a prediction loss term measuring difference between a predicted diagnosis label and ground truth, and wherein machine learning model is trained based on the prediction loss term and the plurality of soft constraints.

20. The non-transitory computer-readable medium of claim 15, wherein the correlation between the pair of diagnosis labels is represented by a correlation matrix.
